(19) United States
(12) Patent Application Publication
(10) Pub. No.: US 2008/0269912 A1
(43) Pub. Date: Oct. 30, 2008

(54) ARTIFICIAL LEG

(75) Inventors: Dieter Gobbers, Heidenheim (DE); Walter Gobbers, Heidenheim (DE)

Correspondence Address: BAKER & DANIELS LLP;111 E. WAYNE STREET, SUITE 800, FORT WAYNE, IN, 46802, US

(73) Assignee: REGO FLORIDA CORPORATION, Lehigh Acres, FL (US)

(21) Appl. No.: 11/740,611

(22) Filed: Apr. 26, 2007

(51) Int. Cl. A61F 2/74; A61F 2/60

(52) U.S. Cl. 623/27; 623/33

(57) ABSTRACT

The invention concerns an artificial leg
with an artificial knee having, for carrying a femoral stump, a joint body which is rotatable about the joint axis;
with a shaft, the upper end of which engages the joint body, and the lower end of which bears an artificial foot;
joint body and shaft cooperate in such a way that a rotational movement of the joint body is transferred into a translative movement of the shaft, and that during the bending of the knee joint the distance between its joint axis and the tip of the artificial foot is reduced.

The invention concerns an artificial leg with an artificial knee and an artificial foot. Such a prosthesis may be attached to a leg's stump above the knee.

Numerous embodiments have become known. FR 2 711 512 A1 describes a prosthesis, by means of which the patient can perform a swinging movement. The swinging movement is controlled, so that a comparably natural walking may be obtained.

Numerous documents address the control of the relative movement of the upper part of an artificial leg, which means the part above the joint prosthesis, with respect to the lower part, which means the part below the joint prosthesis. In many cases, they address the damping of the driving. See GB-A-2 252 503, WO 93/22 991 or WO 92/22 267.

Problems occur during the so-called momentum phase, which means that phase, during which the patient, when striding, pivots the artificial leg in walking direction. Therein, a streaking contact between the artificial foot and the ground occurs. This leads to an insecure walking of the patient.

The invention is based on the task to design an artificial leg of the said kind in such a way that the walking of the patient is improved, especially in the swinging-out phase.

This task is solved by the features of claim 1.

Accordingly, during the bending of the knee joint, the distance between its joint axis and the tip of the artificial foot is reduced. The length of the artificial leg as a whole is hereby reduced during the bending. Thereby, a streaking of the artificial foot or its tip on the floor is avoided. It is further possible to lift only the foot of the artificial leg or the foot's tip. In this context, a further joint is necessary, either between the foot of the artificial leg and the main part of the artificial leg, or the foot's tip and the main part of the foot of the artificial leg.

The invention is further explained with reference to the drawing(s). Therein, in detail the following is shown:

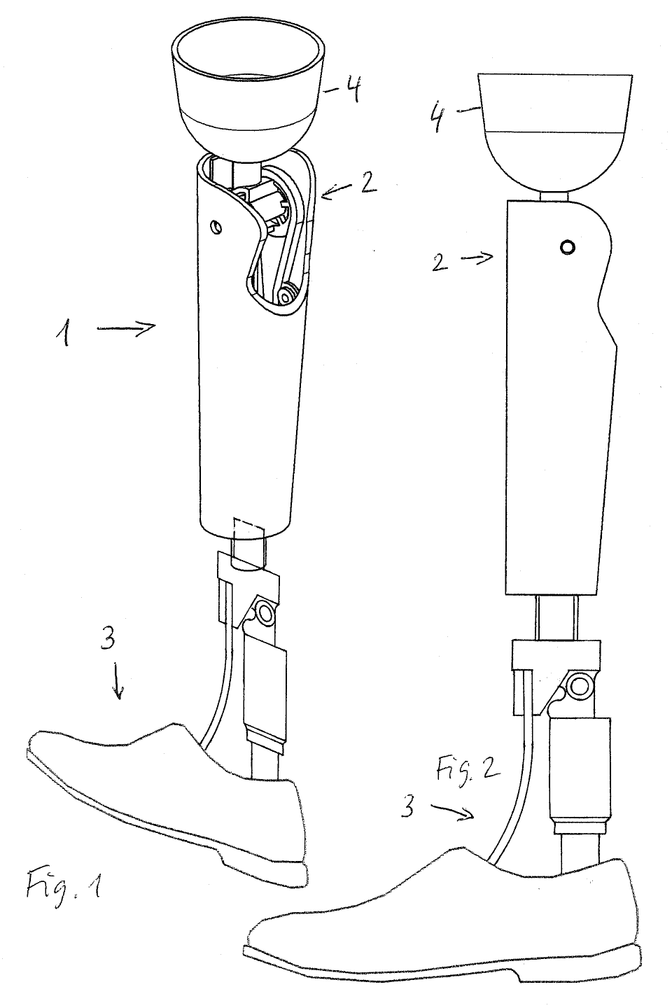

FIG. 1 shows in perspective view an artificial leg with an artificial knee and an artificial foot.

FIG. 2 shows the artificial leg according to FIG. 1 in a side view.

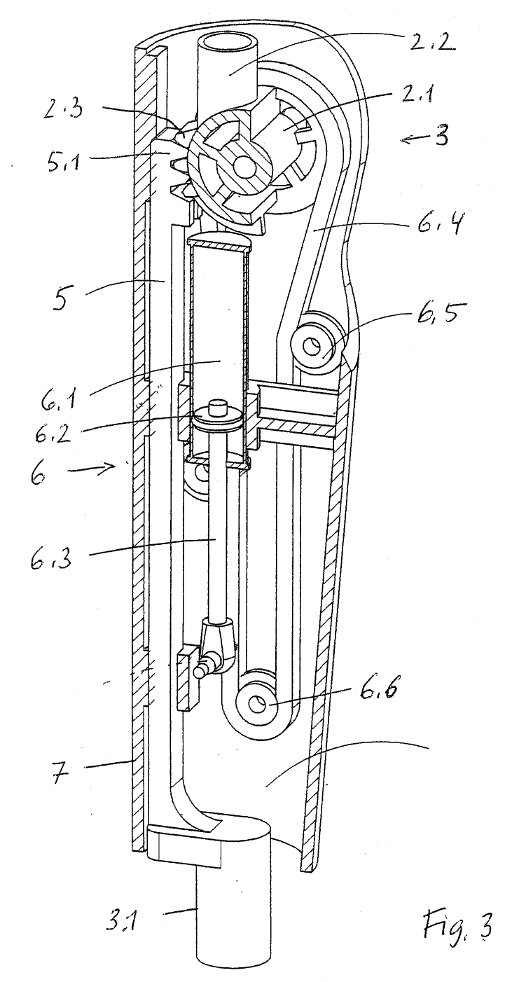

FIG. 3 in perspective view and in a longitudinal sectional view shows the artificial leg according to FIG. 1, but without artificial foot.

FIGS. 4, 5 and 6 show the artificial knee at different bending angles of the knee joint.

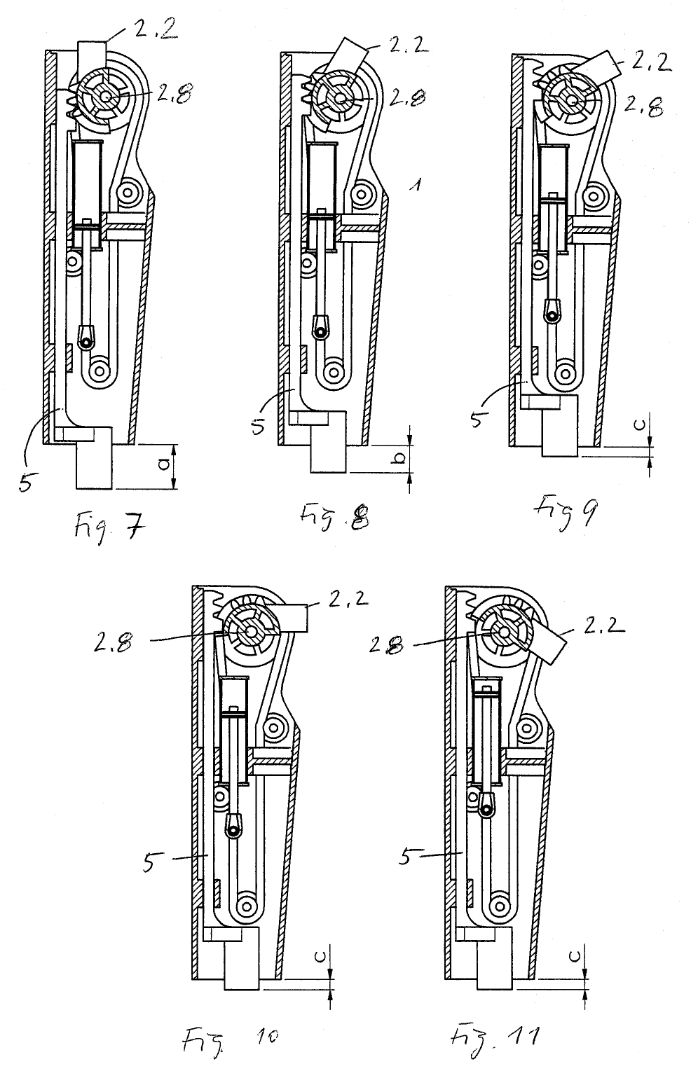

FIGS. 7 to 11 show the artificial leg according to FIG. 1 in a longitudinal section, but without the artificial foot.

FIG. 12 shows a second embodiment of the invention with a control of the lifting movement of the shaft by means of a control surface and a control pulley.

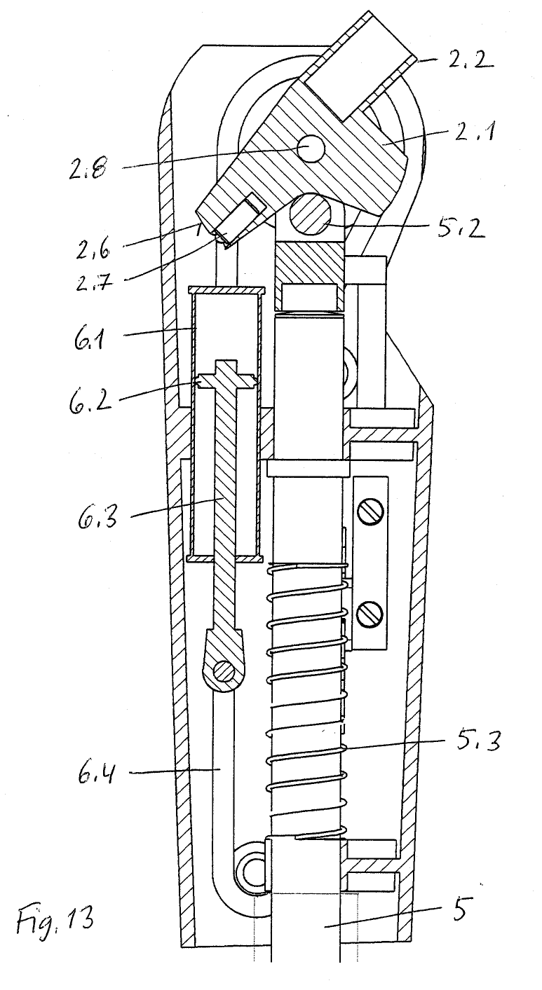

FIG. 13 shows the subject of FIG. 12 after cycling through a bending of the artificial knee 2.

The artificial leg 1 shown in the figures comprises besides others an artificial knee 2 and an artificial foot 3. The artificial knee supports a cup 4 for receiving a femoral stump, which is not shown here.

FIG. 3 shows in detail the construction of the artificial leg, with the exception of the artificial foot. The artificial knee 2 comprises a joint body 2.1, which is supported in a turnable manner. This supports a connection sleeve 2.2, which supports the support cup 4 shown in the FIGS. 1 and 2. Joint body 2.1 is provided with teeth 2.3 at its periphery.

There can further be seen a shaft 5. At its upper end, it is designed as toothed rack and accordingly provided with teeth 5.1. At its lower end, it engages a stud 3.1 of the artificial foot 3.

The teeth 5.1 of the shaft 5 mesh with the teeth 2.3 of the artificial knee 2. A twisting of the joint body 2.1 therefore leads to a vertical displacement movement of the shaft 5, and therefore to a change of the position of the artificial foot 3.

It is further provided a damping device 6. It comprises a hydraulic cylinder 6.1 filled with liquid. A piston 6.2 is relocatable in the hydraulic cylinder 6.1. A piston rod 6.3 engages the piston 6.2.

The damping device 6 further comprises a band 6.4. It entangles the joint body 2.1 as well as two deflection pulleys 6.5, 6.6. The lower end of the piston rod 6.3 is fixed to the band 6.4. The hydraulic cylinder 6.1 is supported by a housing 7 and therefore fixed in position.

If in bending of the artificial knee 2, the joint body 2.1 executes a rotational movement, the band 6.4 will be brought to circulation. The circulation movement of the band 6.4, and thereby also the rotational movement of the joint body 1, will be damped by the damping device 6.

The design of the artificial knee 2 is to be seen in more detail in the FIGS. 4, 5 and 6.

As can be seen, the joint body 2.1 of the artificial knee 2 has only three teeth 2.3 at its periphery.

FIG. 4 shows a state in which the artificial leg 1 is stretched, and the artificial knee 2 therefore is not bent. The teeth 2.3 of the artificial knee 2 and the teeth 5.1 of the shaft 5 therefore are engaged to each other.

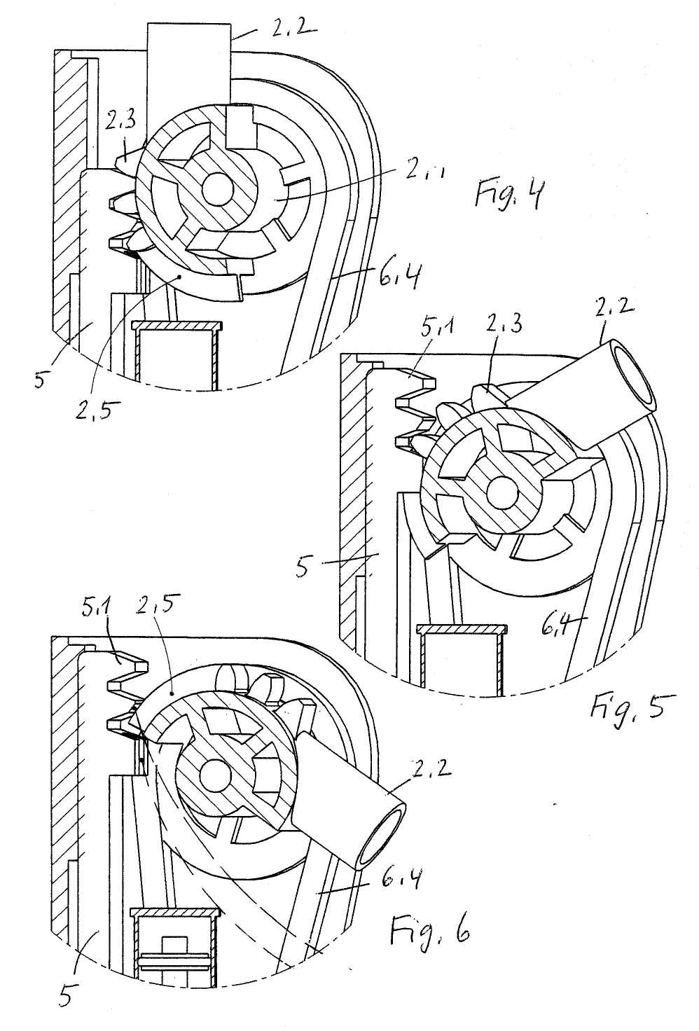

If the artificial knee 2 is bent, the joint body 2.1 describes a clockwise rotational movement. Due to the meshing of teeth between the teeth 2.3 and 5.1, this leads to a lifting of the shaft 5, and this long enough, until the state according to FIG. 5 is reached. This is also to be seen from the position of a connection sleeve 2.2, which is part of the joint body 2.1. This connection sleeve 2.2 is situated in vertical direction at the position according to FIG. 4. At the position according to FIG. 5 it is pivoted by a certain angle though.

If the artificial knee 2 is bent further, and joint body 2.1 is twisted further, the teeth 2.3 and 5.1 are no longer engaged (see FIG. 6). Also after further rotation of the joint body 2.1 in excess of the position shown in FIG. 5, shaft 5 therefore remains in the same height position. In order to avoid a falling down of the shaft 5, a peripheral rib 2.5 is provided, which is part of the joint body 2.1. As can be seen in FIG. 6, the middle one of the teeth 5.1 rests on the peripheral rib 2.5, so that shaft 5 does not slide downwards.

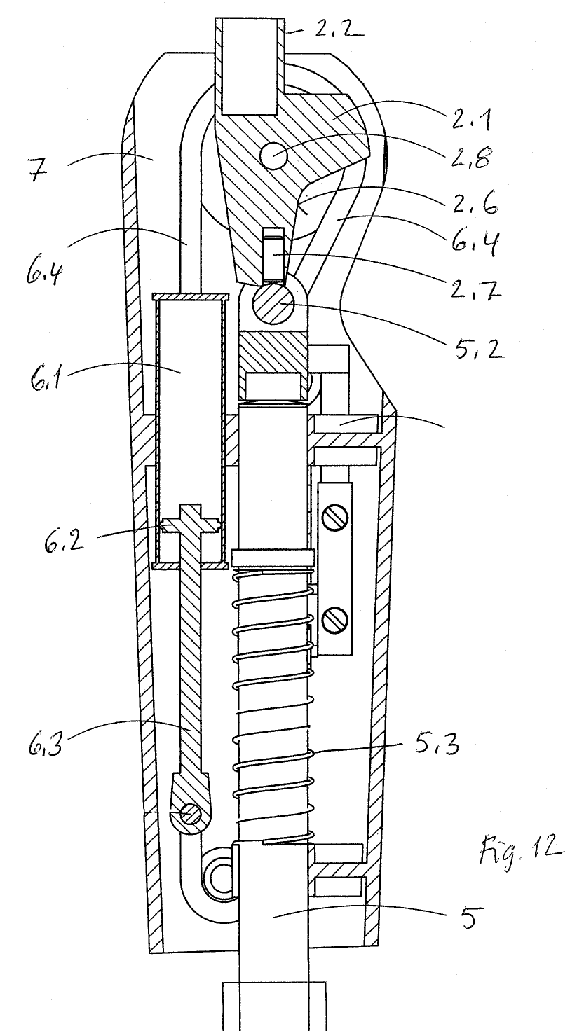

In the FIGS. 7 to 11, five bending positions of the artificial knee 1 are shown. This can again be seen very well from the angular position of the connection sleeve 2.2 of joint body 2.1, as well as from the height position of the stud 3-1 of the artificial foot. As can be seen, the connection sleeve 2.2 of the joint body 2.1, when cycling through, performs the various positions of a clockwise pivoting movement. At the same time, shaft 5 is lifted. At the position according to FIG. 7, it protrudes with respect to the lower edge of the housing 7 by the value of a. This value is then reduced. It becomes value b according to FIG. 8, value c according to FIG. 9, and then remains at this value until the position according to FIG. 11.

Instead of lifting the complete artificial foot 3 when bending by means of the shaft 5, it can also be sufficient to only lift the acute region of the artificial foot 3.

The second embodiment shown in the FIGS. 12 and 13 is generally constructed like the embodiment described before. But the lifting movement of the shaft 5 is controlled by means of a control surface 2.6 of the joint body 2.1 as well as a control roll 5.2 of the shaft 5.

The shaft 5 may comprise a compression spring 5.3. With its lower end, it rests on a flange 7.1 of the housing, and with its upper end on a collar 5.4 of the shaft.

Further, an elastic abutting part 2.7 can be inserted in the joint body 2.1. By means of this construction, a certain latch effect is created, so that the involved components show the tendency to remain in the position according to FIG. 12.

When walking, the control roll 5.2 has to be pressed out of the indentation, in order to allow the knee to fold.

When walking, the prosthesis pivots in forward direction, and in the stretched state of the prosthesis, the control roll 5.2 reaches the range of the indentation of the control surface. The abutting part 2.7 now prevents the control roll from engaging the indentation. When the heel of the prosthesis is put on ground now, the weight of the carrier of the prosthesis now presses onto the prosthesis and presses the control roll into the indentation, so that it rests firmly. The resilient abutting part is compressed, the knee cannot buckle.

During continued walking, the healthy foot is now pivoted forward, and only the foot's tip of the prosthesis that touches the ground. After the prosthesis is no longer loaded by the weight of the carrier, the resilient abutting part of the control roll 5.2 pushes out of its indentation, and the prosthesis knee can buckle and pivot in forward direction.

In the seated position of the patient—with a bending angle of about 90°—the artificial leg is prolonged again, in order to obtain a knee height equal to the healthy leg.

### List of Reference Signs

1 artificial leg
2 artificial knee
2.1 joint body
2.2 connection sleeve
2.3 teeth
2.5 peripheral rib
2.6 control surface
2.7 abutting part
2.8 joint axis
3 artificial foot
3.1 stud
4 support cup
5 shaft
5.1 teeth
5.2 control roll
5.3 compression spring
5.4 collar
6 damping device
6.1 hydraulic cylinder
6.2 piston
6.3 piston rod
6.4 band
6.5 deflection pulley
6.6 deflection pulley
7 housing
7.1 flange

1. Artificial leg,
with an artificial knee having, for carrying a femoral stump, a joint body which is rotatable about the joint's axis;
with a shaft, the upper end of which engages the joint body, and the lower end of which bears an artificial foot;
wherein joint body and shaft cooperate in such a way that a rotational movement of the joint body is transferred into a translative movement of the shaft, and that during the bending of the knee joint the distance between its joint's axis and the tip of the artificial foot is reduced.

2. Artificial leg according to claim 1, characterized by the following features:
the joint body has a toothing at it's periphery;
at its upper end region the shaft is designed as a toothed rack.

3. Artificial leg according to claim 2, characterized in that the number and/or the arrangement of the teeth of joint body or toothed rack are selected in such a way that, when a certain bending angle of the knee joint is exceeded, there results no further lifting of the shaft.

4. Artificial leg according to claim 1, characterized in that the joint body has a curved disk cooperating with the upper end region of the shaft.

5. Artificial leg according to claim 1, characterized by a damping device, which damps the pivoting movement of the shaft.

6. Artificial leg according to claim 5, characterized by the following features:
an endless band loop is provided, which entangles the joint body as well as a deflection pulley, and which is brought into circulation when twisting the joint body;
a hydraulic piston cylinder unit is provided, which damps the circulation movement of the band.

7. Artificial leg according to claim 2, characterized by a damping device, which damps the pivoting movement of the shaft.

8. Artificial leg according to claim 3, characterized by a damping device, which damps the pivoting movement of the shaft.

9. Artificial leg according to claim 4, characterized by a damping device, which damps the pivoting movement of the shaft.
